(19) United States
(12) Patent Application Publication
(10) Pub. No.: US 2025/0161526 A1
(43) Pub. Date: May 22, 2025

(54) EMBOLIC AGENT AND BLOOD VESSEL EMBOLIZATION KIT

(71) Applicant: NATIONAL UNIVERSITY CORPORATION HOKKAIDO UNIVERSITY, Sapporo-shi, Hokkaido (JP)

(72) Inventors: Hailong FAN, Hokkaido (JP); Toshiya OSANAI, Hokkaido (JP); Zhiping JIN, Hokkaido (JP); Takayuki KUROKAWA, Hokkaido (JP); Takayuki NONOYAMA, Hokkaido (JP)

(21) Appl. No.: 18/841,504

(22) PCT Filed: Feb. 27, 2023
(86) PCT No.: PCT/JP2023/007042

§ 371 Date: Aug. 26, 2024

(30) Foreign Application Priority Data
Feb. 28, 2022 (JP) 2022-030098

(51) Int. Cl. A61L 24/06; A61L 24/00

(52) U.S. Cl. CPC A61L 24/06; A61L 24/001; A61L 2430/36

(57) ABSTRACT

The embolic agent according to the present invention includes as an active ingredient a copolymer of a cationic functional group-containing monomer and an aromatic group-containing monomer, the copolymer having at least a part thereof a structure represented by general formula (I) (in the formula, R¹¹ and R¹² are hydrogen atoms or alkyl groups having 1 to 20 carbon atoms which may have a substituent, and satisfy a relational formula: R¹¹=R¹²; Y¹¹ and Y¹² are each independently a single bond or an alkylene group having 1 to 20 carbon atoms; Ar¹¹ is an aromatic hydrocarbon group having 6 to 16 carbon atoms which may have a substituent; X¹¹ is an ammonium group or an amino group; and n11 is an integer of 10 to 1,000.)

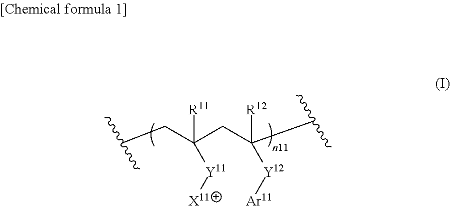

### TECHNICAL FIELD

The present invention relates to an embolic agent and a kit for vascular embolization.

Priority is claimed on Japanese Patent Application No. 2022-030098, filed Feb. 28, 2022, the content of which is incorporated herein by reference.

### BACKGROUND ART

Embolization involves partial or complete occlusion of a blood vessel, restricting the flow of blood through the vessel. Therapeutic embolization is used to treat a variety of conditions, such as cerebral and peripheral aneurysms, arteriovenous malformations, uterine fibroids, and to reduce or block blood flow to tumors. Embolization can be achieved by a variety of means, including the use of polymer microspheres, metal coils, metal or polymer plugs, and liquid embolic materials. Of these means, an appropriate means can be selected based on the size of the blood vessel to be occluded, the desired duration of occlusion, or the type of disease or condition to be treated.

Examples of the liquid embolic materials that have been conventionally used include N-butyl cyanoacrylate (NBCA; trade name: “TRUFILL (registered trademark)” manufactured by Codman & Shurtleff, Inc., etc.) and ethylene vinyl alcohol copolymer (EVOH; trade name: “Onyx (registered trademark)” manufactured by ev3 Endovascular, Inc., etc.) (see, for example, Patent Document 1, etc.). The main mechanism of action of NBCA is a polymerization reaction with blood, and that of EVOH is precipitation in blood and coagulation action to embolize blood vessels.

As an alternative, the inventors have developed a cationic π polymer (poly(cation-adj-π)) having an adjacent cationic functional group-aromatic group arrangement (see, for example, Non-Patent Document 1, etc.). It has been reported that the cationic 7 polymer has strong yet reversible adhesion to a negatively charged surface in an aqueous solution (0.7 M NaCl aqueous solution) containing the same amount of salt as seawater.

### CITATION LIST

### Patent Documents

Patent Document 1: Published Japanese Translation No. 2000-517298 of the PCT International Publication

### Non-Patent Documents

Non-Patent Document 1: Fan H et al., “Adjacent cationic-aromatic sequences yield strong electrostatic adhesion of hydrogels in seawater.”, NATURE COMMUNICATIONS, Vol. 10, No. 5127, 2019.
Non-patent Document 2: “Approval Review Report (New Medical Devices)”, issued by the Pharmaceuticals and Medical Devices Agency (PMDA), general name: Other tube and catheter-related devices (vascular embolization set 100420997), Aug. 1, 2008.

### SUMMARY OF INVENTION

### Technical Problem

However, with the conventional liquid embolic materials such as NBCA and EVOH, the embolic material may adhere to the tip of the catheter, and when the catheter is withdrawn, there is a risk of damaging the inner wall of the blood vessel or the catheter insertion site, causing bleeding. EVOH requires the use of dimethyl sulfoxide (DMSO) as a solvent, and the approval review report (new medical device) (Non-Patent Document 2) issued by the Pharmaceuticals and Medical Devices Agency (PMDA) describes the possibility of local nerve damage and damage to the blood vessel wall caused by DMSO for commercially available EVOH products. For this reason, only an amount of EVOH that can be dissolved in an amount of DMSO that does not cause neurotoxicity can be administered, and it may be necessary to administer it two or more times. Furthermore, EVOH requires the use of a special catheter and syringe suitable for using DMSO in order to administer it, which places a heavy burden on the patient in terms of cost.

The present invention has been made in consideration of the above-described circumstances, and provides an embolic agent that has vascular embolization properties equivalent to those of conventional embolic agents, while being easy to inject with a syringe and having excellent safety and stability in the living body, and a kit for vascular embolization including the embolic agent.

### Solution to Problem

As a result of intensive research into achieving the above-mentioned object, the inventors have discovered that a copolymer having, at least in part, a structure consisting of an alternating arrangement of cationic functional group-containing monomer units and aromatic group-containing monomer units has good injectability using a syringe and generates almost no resistance when a catheter is withdrawn from within a blood vessel, and that administration of the copolymer into a blood vessel forms a safe and stable blood clot-like gel mass through electrostatic interaction with blood components, thereby completing the present invention.

That is, the present invention includes the following aspects.
(1) An embolic agent including, as an active ingredient, a copolymer of a cationic functional group-containing monomer and an aromatic group-containing monomer, the copolymer having at least a part thereof a structure represented by the following general formula (I):

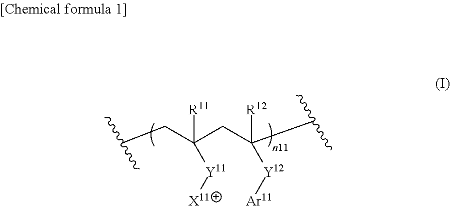

(in general formula (I), R¹¹ and R¹² are hydrogen atoms or alkyl groups having 1 to 20 carbon atoms which may have a substituent, and satisfy a relational formula: R¹¹=R¹²; Y¹¹ and Y¹² are each independently a single bond, or an alkylene group having 1 to 20 carbon atoms which may contain one or more selected from the group consisting of a hydroxyl group, an ester bond, an ether bond, a sulfide group, a carbonyl group, an amide bond and a phosphodiester bond; Ar¹¹ is an aromatic hydrocarbon group having 6 to 16 carbon atoms which may have a substituent; X¹¹ is an ammonium group or an amino group; and n11 is an integer of 10 to 1,000.)
(2) The embolic agent according to (1), wherein in general formula (I), Ar¹¹ is a phenyl group which may have a substituent.
(3) The embolic agent according to (1) or (2), wherein in general formula (I), X¹¹ is a quaternary ammonium group.
(4) The embolic agent according to any one of (1) to (3), wherein in general formula (I), an absolute value of a difference between a number of carbon atoms of Y¹² and a number of carbon atoms of Y¹¹ is 0 or more and 3 or less.
(5) The embolic agent according to any one of (1) to (4), wherein the structure represented by general formula (I) is a structure represented by the following general formula (I-1):

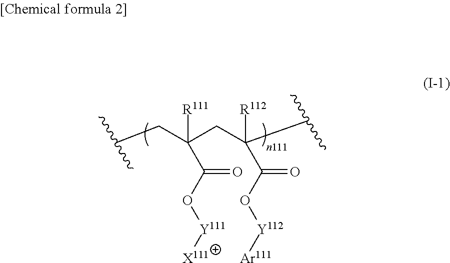

(in general formula (I-1), R¹¹¹ and R¹¹² are hydrogen atoms or methyl groups, and satisfy a relational formula: R¹¹¹=R¹¹²; Y¹¹¹ and Y¹¹² are each independently an alkylene group having 1 to 20 carbon atoms which may contain one or more selected from the group consisting of a hydroxyl group, an ether bond, a sulfide group and a phosphodiester bond; Ar¹¹¹ is a phenyl group which may have a substituent; X¹¹¹ is a quaternary ammonium group; and n111 is an integer of 10 to 1,000.)
(6) The embolic agent according to any one of (1) to (5), wherein in the copolymer, a molar ratio of a unit derived from the cationic functional group-containing monomer to a unit derived from the aromatic group-containing monomer is 1:4 to 4:1.
(7) A kit for vascular embolization including the embolic agent according to any one of (1) to (6) and a contrast agent.

### Advantageous Effects of Invention

According to the above-described embodiment of the embolic agent, it is possible to provide an embolic agent that has vascular embolization properties equivalent to those of conventional embolic agents, while being easy to inject with a syringe and excellent in safety and stability in the living body.

### BRIEF DESCRIPTION OF DRAWINGS

FIG. 1A is a graph showing the NMR spectrum in Example 1.

FIG. 1B is a graph showing the NMR spectrum in Example 1.

FIG. 1C is a graph showing the NMR spectrum in Example 1.

FIG. 1D is a graph showing the NMR spectrum in Example 1.

FIG. 1E is a graph showing the copolymerization rate of the cationic functional group-containing monomer (ATAC) and the aromatic group-containing monomer (PEA) in Example 1.

FIG. 2A is a diagram showing the protocol of the qualitative blood agglomeration test in Example 2.

FIG. 2B is an image showing the results of the qualitative blood agglomeration test in Example 2.

FIG. 3 is a graph showing the results of the quantitative blood agglomeration test in Example 2.

FIG. 4 is a graph showing the results of the rheological test in Example 2.

FIG. 5A is a schematic diagram of a syringe used in the injection force test in Example 3.

FIG. 5B is a graph showing the results of the injection force test using a syringe in Example 3.

FIG. 5C is a graph showing the results of the injection force test using a syringe in Example 3.

FIG. 6A is a schematic diagram of a syringe and a microcatheter used in the injection force test in Example 3.

FIG. 6B is a graph showing the results of an injection force test using a syringe and a microcatheter in Example 3.

FIG. 7A is an image (left) and a schematic diagram (right) of the device used in the traction force test in Example 4.

FIG. 7B is a graph showing the results of the traction force test in Example 4.

FIG. 7C is a graph showing the results of the traction force test in Example 4.

FIG. 8A is a graph showing the results of the biochemical test in Example 5.

FIG. 8B is a hematoxylin and eosin (H&E) stained image of a tissue section at the implantation site of the polymer hydrogel in Example 5.

FIG. 9 shows a visual observation image (upper) and a thermography image (lower) of the hind legs in Example 6.

FIG. 10 shows a bright field image (left side), an H&E stained image (middle side), and an acid blue stained image (right side) of a tissue section around the femoral artery of a hind limb into which a polymer was injected in Example 6.

FIG. 11 is a computed tomography (CT) image in Example 7.

### DESCRIPTION OF EMBODIMENTS

### <<Embolizing Agents>>

The embolic agent of the present embodiment contains as an active ingredient a copolymer of a cationic functional group-containing monomer and an aromatic group-containing monomer, the copolymer having at least a part thereof a structure represented by the following general formula (I) (hereinafter, sometimes referred to as “structure (I)”).

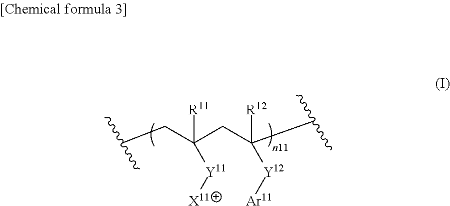

(In general formula (I), R¹¹ and R¹² are hydrogen atoms or alkyl groups having 1 to 20 carbon atoms which may have a substituent, and satisfy a relational formula: R¹¹=R¹². Y¹¹ and Y¹² are each independently a single bond, or an alkylene group having 1 to 20 carbon atoms which may contain one or more selected from the group consisting of a hydroxyl group, an ester bond, an ether bond, a sulfide group, a carbonyl group, an amide bond, and a phosphodiester bond. Ar¹¹ is an aromatic hydrocarbon group having 6 to 16 carbon atoms which may have a substituent. X¹¹ is an ammonium group or an amino group. n11 is an integer of 10 to 1,000. The wavy line represents a bonding site.)

The embolic agent of the present embodiment, having the above-mentioned configuration, has vascular embolization properties equivalent to those of conventional embolic agents, while being excellent in injectability with a syringe, and in safety and stability in the living body.

In this specification, “containing as an active ingredient” means containing a therapeutically effective amount of the copolymer. In this specification, “therapeutically effective amount” means an amount of the copolymer, or a combination of the copolymer and one or more active agents, that induces the biological or medical effect or response desired by a physician, clinician, veterinarian, researcher, or other appropriate professional when administered according to a desired treatment regimen. A preferred therapeutically effective amount is an amount that improves the symptoms of a disease requiring intravascular embolization. Specific examples of such diseases are described below.

### <Copolymer>

The copolymer is formed by copolymerizing a cationic functional group-containing monomer and an aromatic group-containing monomer, and has the above-described structure (I) at least in part. The structure (I) is consisting of an arrangement in which the cationic functional group-containing monomer and the aromatic group-containing monomer are alternately arranged. Alternatively, the structure (I) can be said to be a structure in which units each consisting of one molecule of the cationic functional group-containing monomer and one molecule of the aromatic group-containing monomer are continuous.

The mechanism of blood coagulation by the copolymer is that negatively charged blood components (red blood cells, white blood cells, platelets and the like) or proteins in the blood bind to the cationic functional groups in the copolymer via electrostatic interaction to form a blood clot-like gel mass (hereinafter referred to as blood gel) containing blood, thereby embolizing the blood vessel. It is presumed that the aromatic groups in the blood gel form a hydrophobic field, stabilizing the blood gel in the body. Note that the above-described mechanism is merely an example for the embolic agent of the present embodiment, and the present invention is not limited to the above-described mechanism as long as it can exert the effect.

[R¹¹ and R¹²]

R¹¹ and R¹² are hydrogen atoms or alkyl groups having 1 to 20 carbon atoms which may have a substituent. In addition, a formula: R¹¹=R¹² is satisfied. In the present invention and the present specification, the “alkyl group having 1 to 20 carbon atoms which may have a substituent” includes both an “alkyl group having 1 to 20 carbon atoms which has no substituent” and an “alkyl group having 1 to 20 carbon atoms which has a substituent”. By using a cationic functional group-containing monomer and an aromatic group-containing monomer which satisfy the formula, a copolymer having the structure (I) at least in a part thereof can be obtained.

The “alkyl group having 1 to 20 carbon atoms” for R¹¹ and R¹² is preferably a chain-like group. The chain alkyl group may be linear or branched. The chain alkyl group is preferably a group having 1 to 6 carbon atoms, and specific examples thereof include a methyl group, an ethyl group, a propyl group, an isopropyl group, a butyl group, an isobutyl group, a tert-butyl group, a pentyl group, a hexyl group and the like.

Examples of the substituent on R¹¹ and R¹² include halogen atoms and the like. Examples of the halogen atoms include a fluorine atom, a chlorine atom, a bromine atom, an iodine atom and the like. The number of the substituents possessed by R¹¹ and R¹² may be 1 or more, preferably 1 or more and 5 or less, and more preferably 1 or more and 3 or less.

Among these, R¹¹ and R¹² are preferably a hydrogen atom or an alkyl group having 1 to 10 carbon atoms, more preferably a hydrogen atom, a methyl group, or an ethyl group, and even more preferably a hydrogen atom or an ethyl group.

[Y¹¹ and Y¹²]

Y¹¹ and Y¹² are each independently a single bond, or an alkylene group having 1 to 20 carbon atoms which may contain one or more selected from the group consisting of a hydroxyl group, an ester bond, an ether bond, a sulfide group, a carbonyl group, an amide bond, and a phosphodiester bond. In the present invention and the present specification, the “alkylene group having 1 to 20 carbon atoms which may contain one or more selected from the group consisting of a hydroxyl group, an ester bond, an ether bond, a sulfide group, a carbonyl group, an amide bond, and a phosphodiester bond” includes both an “alkylene group having 1 to 20 carbon atoms which does not contain one or more selected from the group consisting of a hydroxyl group, an ester bond, an ether bond, a sulfide group, a carbonyl group, an amide bond, and a phosphodiester bond” and a “single bond or an alkylene group having 1 to 20 carbon atoms which contains one or more selected from the group consisting of a hydroxyl group, an ester bond, an ether bond, a sulfide group, a carbonyl group, an amide bond, and a phosphodiester bond”. The “alkylene group having 1 to 20 carbon atoms which contains one or more selected from the group consisting of a hydroxyl group, an ester bond, an ether bond, a sulfide group, a carbonyl group, an amide bond, and a phosphodiester bond” is preferably an alkylene group having 1 to 20 carbon atoms which contains one to three selected from the group consisting of a hydroxyl group, an ester bond, an ether bond, a sulfide group, a carbonyl group, an amide bond, and a phosphodiester bond.

Examples of the alkylene group having 1 to 20 carbon atoms for Y¹¹ and Y¹² include a methylene group, an ethylene group, a propylene group, a butylene group, a pentylene group, a hexylene group, a heptylene group, an octylene group, a nonylene group, a decylene group, an undecylene group, a dodecylene group, a tetradecylene group, a hexadecylene group, an octadecylene group, a nonadecylene group, an icosylene group and the like. Among these, the alkylene group for Y¹¹ and Y¹² is preferably a methylene group, an ethylene group, a propylene group, a butylene group, a pentylene group, a hexylene group, a heptylene group, an octylene group, a nonylene group, or a decylene group, more preferably a methylene group, an ethylene group, a propylene group, a butylene group, a pentylene group, a hexylene group, a heptylene group, or an octylene group, and even more preferably a methylene group, an ethylene group, a propylene group, or a butylene group.

Among these, Y¹¹¹ and Y¹² are preferably an alkylene group having 1 to 10 carbon atoms, which may contain one or more, preferably one to three, selected from the group consisting of a hydroxyl group, an ester bond, an ether bond, a sulfide group, and a phosphodiester bond.

In addition, in view of the ease of interaction between the cationic functional group and the aromatic group, it is preferable that the lengths of the linkers Y¹¹ and Y¹² are approximately the same so that the distance between these functional groups is short, and it is more preferable that the absolute value of the difference between the number of carbon atoms of Y¹¹ and the number of carbon atoms of Y¹² is 0 or more and 3 or less.

More specifically, the preferred examples of Y¹¹ and Y¹² include groups represented by the following general formulas (Iy-1) to (Iy-7) (hereinafter, sometimes referred to as “group (Iy-1)” or the like). Note that groups (Iy-1) to (Iy-7) are merely examples of preferred Y¹¹ and Y¹², and preferred Y¹¹ and Y¹² are not limited thereto. In groups (Iy-1) to (Iy-7), the wavy line on the left side represents a bonding site with the carbon atom to which R¹¹¹ or R¹¹² is bonded, and the wavy line on the right side represents a bonding site with X¹¹ or Ar¹¹.

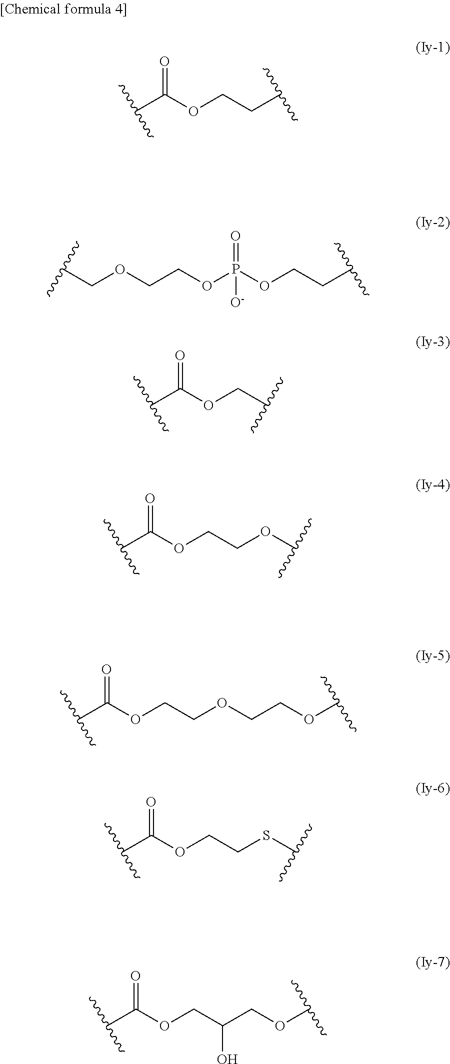

### [Ar¹¹]

Ar¹¹ is an aromatic hydrocarbon group having 6 to 16 carbon atoms which may have a substituent. Examples of the aromatic hydrocarbon group having 6 to 16 carbon atoms for Ar¹¹ include a phenyl group, a naphthyl group, an anthracenyl group and the like. In the present invention and this specification, the “aromatic hydrocarbon group having 6 to 16 carbon atoms which may have a substituent” includes both an “aromatic hydrocarbon group having 6 to 16 carbon atoms which does not have a substituent” and an “aromatic hydrocarbon group having 6 to 16 carbon atoms which has a substituent”.

Examples of the substituent on Ar¹¹ include an alkyl group, a halogen atom and the like. Examples of the alkyl group and the halogen atom include the same as those exemplified above for R¹¹ and R¹². When Ar¹¹ is an “aromatic hydrocarbon group having 6 to 16 carbon atoms which has a substituent”, the number of the substituents in the aromatic hydrocarbon group is preferably 1 to 5, and more preferably 1 to 3.

Among these, Ar¹¹ is preferably a phenyl group which may have a substituent, and more preferably a phenyl group which does not have a substituent or a phenyl group which has 1 to 3 substituents.

### [X¹¹]

X¹¹ is a cationic functional group, and is an ammonium group or an amino group. Among these, X¹¹ is preferably an ammonium group, and more preferably a quaternary ammonium group.

Preferred examples of the quaternary ammonium group include groups represented by the following general formulas (Ix-1) to (Ix-7) (hereinafter, sometimes referred to as “group (Ix-1)” or the like). Note that groups (Ix-1) to (Ix-7) are merely examples of the preferred quaternary ammonium groups, and the preferred quaternary ammonium groups are not limited thereto. The wavy line represents a bonding site with Y¹¹.

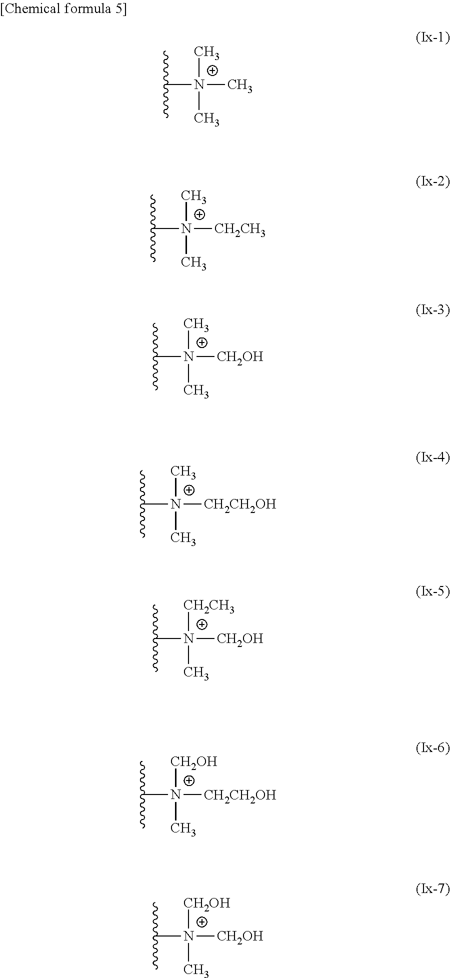

The quaternary ammonium group is usually supplied in an ionically bonded state with a suitable anion. Examples of the anions that can be ionically bonded with the quanternary ammonium group include halogen ions such as a chloride ion, a bromide ion, a fluoride ion, an iodide ion or the like, a hydroxide ion, a sulfate ion, an acetate ion and the like, among which a chloride ion is preferred.

[n11]

n11 is an integer of 10 or more and 1,000 or less, preferably an integer of 30 or more and 1,000 or less, more preferably an integer of 50 or more and 1,000 or less, even more preferably an integer of 70 or more and 1,000 or less, and particularly preferably an integer of 100 or more and 1,000 or less.

In the structure (I), the wavy line on the cationic functional group-containing monomer unit side and the wavy line on the aromatic group-containing monomer unit side each independently represent a bonding site with any one of the following:
1) A hydrogen atom;
2) A unit derived from a polymerization initiator;
3) One or more molecules of cationic functional group-containing monomer units in the case of the wavy line on the cationic functional group-containing monomer unit side;
4) One or more molecules of aromatic group-containing monomer units in the case of the wavy line on the aromatic group-containing monomer unit side.

The copolymer may have one or more structures (I). That is, when the cationic functional group-containing monomer unit is A and the aromatic group-containing monomer unit is B, the copolymer may have, for example, the following sequence. The following sequence is merely an example of the sequence of the copolymer, and the sequence of the copolymer is not limited to the following. In the following sequence, H represents a hydrogen atom, M represents a unit derived from a polymerization initiator, n11 is the same as n11 described above, and n12 and n13 are each independently an integer of 1 or more.
1) H-(AB)ₙ₁₁-H;
2) M-(AB)ₙ₁₁-H;
3) H-(AB)ₙ₁₁-M;
4) M-(AB)ₙ₁₁-M;
5) A-(AB)ₙ₁₁-B;
6) A-(AB)ₙ₁₁-(B)ₙ₁₂-(AB)ₙ₁₁-B;
7) A-(AB)ₙ₁₁-(B)ₙ₁₂-(A)ₙ₁₃-(AB)ₙ₁₁-B

The preferred examples of the structure (I) include a structure represented by the following general formula (I-1) (hereinafter, sometimes referred to as “structure (I-1)”). Note that the structure (I-1) is merely an example of the preferred structure (I), and the preferred structure (I) is not limited thereto.

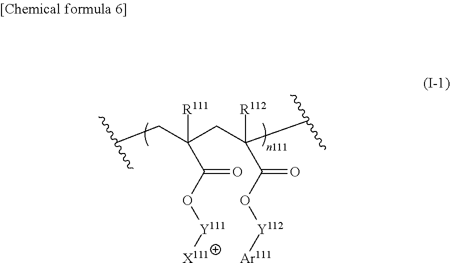

(In general formula (I-1), R¹¹¹ and R¹¹² are hydrogen atoms or methyl groups, and satisfy a relational formula: R¹¹¹=R¹¹². Y¹¹¹ and Y¹¹² are each independently an alkylene group having 1 to 20 carbon atoms which may contain one or more selected from the group consisting of a hydroxyl group, an ether bond, a sulfide group, and a phosphodiester bond. Ar¹¹¹ is a phenyl group which may have a substituent. X¹¹¹ is a quaternary ammonium group. n111 is an integer of 10 to 1,000. The wavy line represents a bonding site.)

[R¹¹¹ and R¹¹²]

R¹¹¹ and R¹¹² are a hydrogen atom or a methyl group and satisfy a relational formula: R¹¹¹=R¹¹².

[Y¹¹¹ and Y¹¹²]

Y¹¹¹ and Y¹¹² are each independently an alkylene group having 1 to 20 carbon atoms which may contain one or more selected from the group consisting of a hydroxyl group, an ether bond, a sulfide group, and a phosphodiester bond. In the present invention and the present specification, an “alkylene group having 1 to 20 carbon atoms which may contain one or more selected from the group consisting of a hydroxyl group, an ether bond, a sulfide group, and a phosphodiester bond” includes both an “alkylene group having 1 to 20 carbon atoms which does not contain one or more selected from the group consisting of a hydroxyl group, an ether bond, a sulfide group, and a phosphodiester bond” and an “alkylene group having 1 to 20 carbon atoms which contains one or more selected from the group consisting of a hydroxyl group, an ether bond, a sulfide group, and a phosphodiester bond”. The “alkylene group having 1 to 20 carbon atoms which contains one or more selected from the group consisting of a hydroxyl group, an ether bond, a sulfide group, and a phosphodiester bond” is preferably an alkylene group having 1 to 20 carbon atoms which contains one to three selected from the group consisting of a hydroxyl group, an ether bond, a sulfide group, and a phosphodiester bond.

Examples of the alkylene group having 1 to 20 carbon atoms for Y¹¹¹ and Y¹¹² include the same groups as those exemplified above for Y¹¹ and Y¹².

Among these, Y¹¹¹ and Y¹¹² are preferably an alkylene group having 1 to 10 carbon atoms which may contain one or more, preferably one to three, selected from the group consisting of a hydroxyl group, an ether bond, a sulfide group, and a phosphodiester bond; more preferably an alkylene group having 1 to 6 carbon atoms which may contain one or more, preferably one to three, selected from the group consisting of a hydroxyl group, an ether bond, a sulfide group, and a phosphodiester bond; even more preferably an alkylene group having 1 to 4 carbon atoms which may contain one or more selected from the group consisting of a hydroxyl group, an ether bond, a sulfide group, and a phosphodiester bond; and still even more preferably an alkylene group having 1 to 4 carbon atoms which does not contain one or more selected from the group consisting of a hydroxyl group, an ether bond, a sulfide group, and a phosphodiester bond, or an alkylene group having 1 to 4 carbon atoms which contains one to three selected from the group consisting of a hydroxyl group, an ether bond, a sulfide group, and a phosphodiester bond.

### [Ar¹¹¹]

Ar¹¹¹ is a phenyl group which may have a substituent. Examples of the substituent include the same as those exemplified above for Ar¹¹. Ar¹¹¹ is preferably a phenyl group which does not have a substituent or a phenyl group which has 1 to 3 substituents.

### [X¹¹¹]

X¹¹¹ is a quaternary ammonium group. Examples of the quaternary ammonium group include the same groups as those exemplified above for X¹¹.

[n111]

n111 is an integer of 10 or more and 1,000 or less, preferably an integer of 30 or more and 1,000 or less, more preferably an integer of 50 or more and 1,000 or less, even more preferably an integer of 70 or more and 1,000 or less, and particularly preferably an integer of 100 or more and 1,000 or less.

In the structure (I-1), the wavy line on the cationic functional group-containing monomer unit side and the wavy line on the aromatic group-containing monomer unit side are the same as the wavy line on the cationic functional group-containing monomer unit side and the wavy line on the aromatic group-containing monomer unit side in the structure (I), respectively.

The preferred examples of the structure (I-1) include structures represented by the following general formulas (I-1-1) to (I-1-6). Note that the structures (I-1-1) to (I-1-6) are merely examples of the preferred structure (I-1), and the preferred structure (I-1) is not limited thereto.

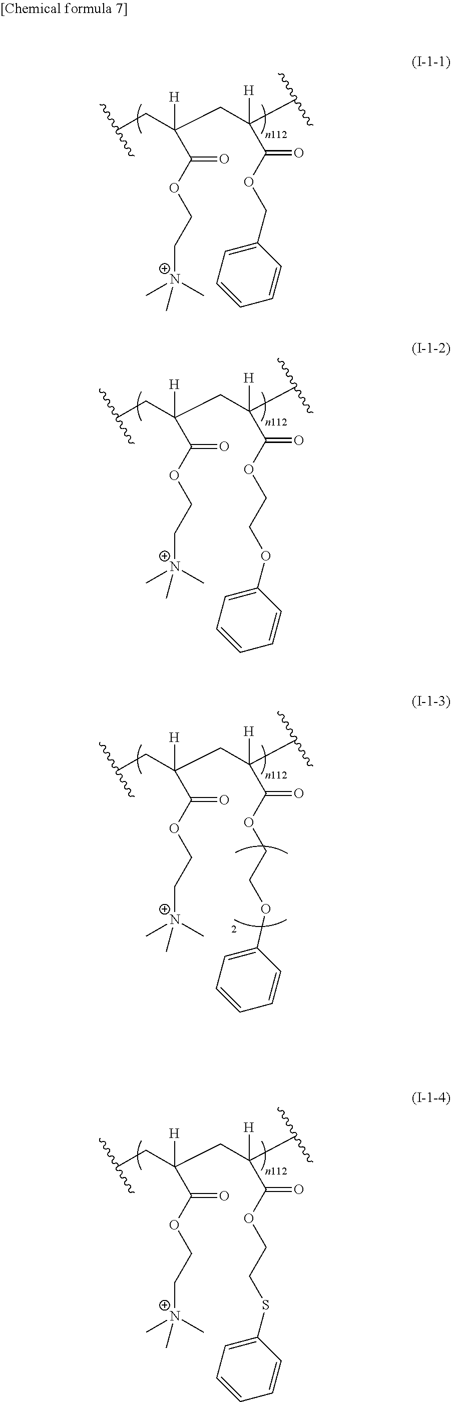

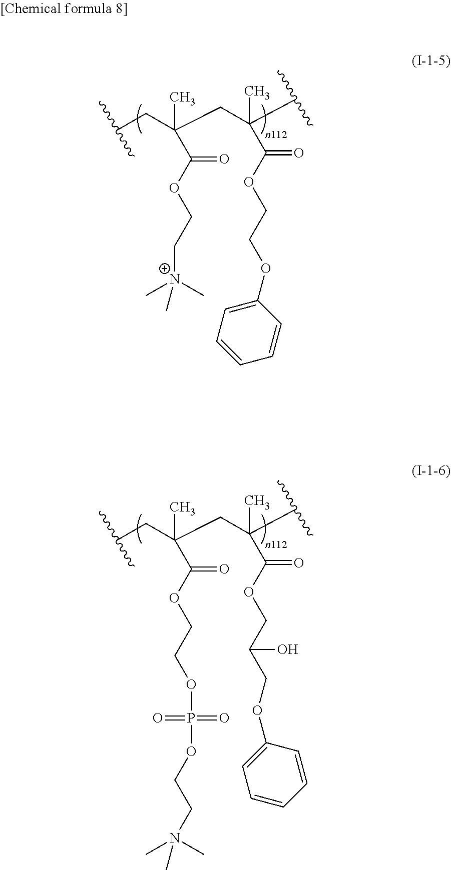

(In the above general formula, n112 is the same as n111 described above.)

### [Cationic Functional Group-Containing Monomer]

The cationic functional group-containing monomer may be any monomer having a cationic functional group and a polymerizable functional group, and examples thereof include a compound represented by the following general formula (Ia) (hereinafter, sometimes referred to as “compound (Ia)”):

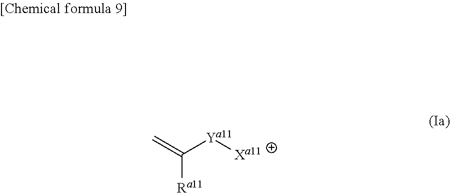

(In formula (Ia), R^{a11}, Y^{a11} and X^{a11} are the same as R¹¹, Y¹¹, and X¹¹, respectively, described above.)

The preferred examples of compound (Ia) include a compound represented by the following general formula (Ia-1) (hereinafter, may be referred to as “compound (Ia-1)”). Compound (Ia-1) is a monomer having a (meth)acrylic acid ester skeleton. Note that compound (Ia-1) is merely an example of the preferred compound (Ia), and the preferred compound (Ia) is not limited thereto.

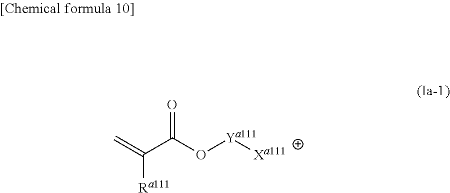

(In formula (Ia-1), R^{a111}, Y^{a111}, and X^{a111} are the same as R¹¹¹, Y¹¹¹, and X¹¹¹, respectively, described above.)

The preferred examples of compound (Ia-1) include compounds represented by the following general formulas (Ia-1-1) to (Ia-1-4) (hereinafter, sometimes referred to as “compound (Ia-1-1) and the like”). Compounds (Ia-1-1) to (Ia-1-4) are monomers having a (meth)acrylic acid ester skeleton. Note that compounds (Ia-1-1) to (Ia-1-4) are merely examples of the preferred compound (Ia-1), and the preferred compound (Ia-1) is not limited thereto.

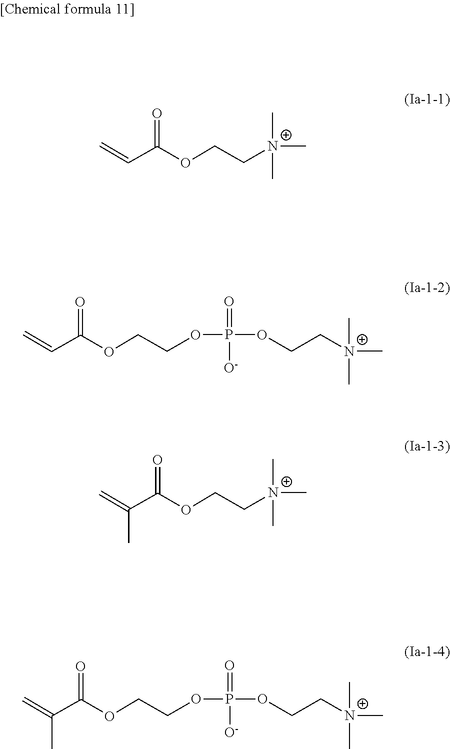

Compound (Ia-1-1) is 2-(acryloyloxy)ethyl trimethylammonium.

Compound (Ia-1-2) is 2-(acryloyloxy)ethyl 2-(trimethylammonio)ethyl phosphate.

Compound (Ia-1-3) is 2-(metacryloxy)ethyl trimethylammonium.

Compound (Ia-1-4) is 2-(metacryloxy)ethyl 2-(trimethylammonio)ethyl phosphate.

### [Aromatic Group-Containing Monomer]

The aromatic group-containing monomer may be any monomer having an aromatic group and a polymerizable functional group, and examples thereof include a compound represented by the following general formula (Ib) (hereinafter, sometimes referred to as “compound (Ib)”).

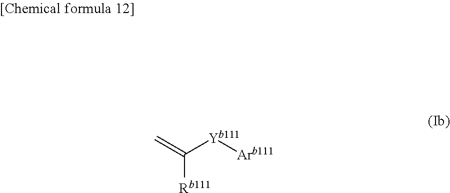

(In formula (Ib), R^{b11}, Y^{b11}, and Ar^{b11} are the same as R¹², Y¹², and Ar¹¹, respectively, described above.)

The preferred examples of compound (Ib) include a compound represented by the following general formula (Ib-1) (hereinafter, may be referred to as “compound (Ib-1)”). Compound (Ib-1) is a monomer having a (meth)acrylic acid ester skeleton. Note that compound (Ib-1) is merely an example of preferred compound (Ib), and the preferred compound (Ib) is not limited thereto.

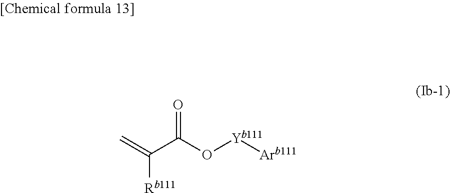

(In general formula (Ib-1), R^{b111}, Y^{b111} and Ar^{b111} are the same as R¹¹², Y¹¹² and Ar¹¹¹, respectively, described above.)

The preferred examples of compound (Ib-1) include compounds represented by the following general formulas (Ib-1-1) to (Ib-1-10) (hereinafter, sometimes referred to as “compound (Ib-1-1) and the like”). Compounds (Ib-1-1) to (Ib-1-10) are monomers having a (meth)acrylic acid ester skeleton. Compounds (Ib-1-1) to (Ib-1-10) are merely examples of the preferred compound (Ib-1), and the preferred compound (Ib-1) is not limited thereto.

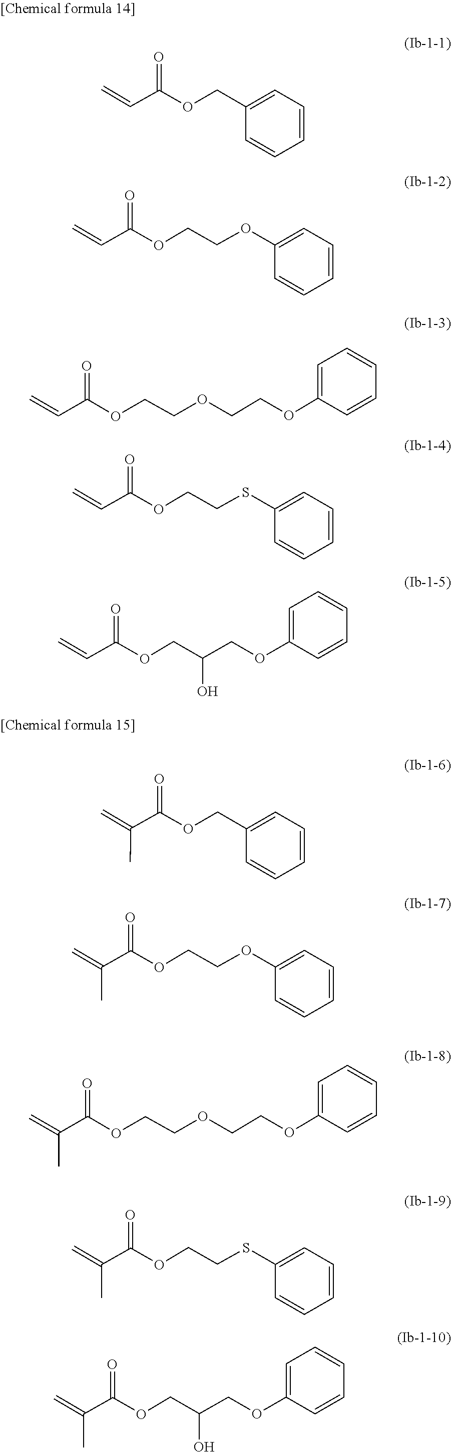

Compound (Ib-1-1) is benzyl acrylate.

Compound (Ib-1-2) is 2-phenoxyethyl acrylate (PEA).

Compound (Ib-1-3) is 2-(2-phenoxyethoxy)ethyl acrylate.

Compound (Ib-1-4) is 2-(phenylsulfanyl) ethyl acrylate.

Compound (Ib-1-5) is 2-hydroxy-3-phenoxypropyl acrylate.

Compound (Ib-1-6) is benzyl methacrylate.

Compound (Ib-1-7) is 2-phenoxyethyl methacrylate.

Compound (Ib-1-8) is 2-(2-phenoxyethoxy)ethyl methacrylate.

Compound (Ib-1-9) is 2-(phenylsulfanyl)ethyl methacrylate.

Compound (Ib-1-10) is 2-hydroxy-3-phenoxypropyl methacrylate.

### [Other Monomers]

The copolymer may have units derived from other monomers in addition to the units derived from the cationic functional group-containing monomer and the aromatic group-containing monomer.

The other monomers may be any monomers that do not have a cationic functional group or an aromatic group, but have a polymerizable functional group that can be polymerized with the cationic functional group-containing monomer and the aromatic group-containing monomer. Examples of such other monomers include the following. These may be used alone or in combination of two or more.
(i) (Meth)acrylic acid esters such as methyl (meth)acrylate, ethyl (meth)acrylate, propyl (meth)acrylate, isopropyl (meth)acrylate, N-butyl (meth)acrylate, isobutyl (meth)acrylate, sec-butyl (meth)acrylate, t-butyl (meth)acrylate, pentyl (meth)acrylate, isopentyl (meth)acrylate, hexyl (meth)acrylate, 2-ethylhexyl (meth)acrylate, heptyl (meth)acrylate, octyl (meth)acrylate, isooctyl (meth)acrylate, nonyl (meth)acrylate, isononyl (meth)acrylate, decyl (meth)acrylate, isodecyl (meth)acrylate, undecyl (meth)acrylate, dodecyl (meth)acrylate (lauryl (meth)acrylate), tridecyl (meth)acrylate, tetradecyl (meth)acrylate, pentadecyl (meth)acrylate, hexadecyl (meth)acrylate, heptadecyl (meth)acrylate, stearyl (meth)acrylate, isostearyl (meth)acrylate, nonadecyl (meth)acrylate, eicosyl (meth)acrylate or the like.
(ii) (Meth)acrylic acid esters having a hydroxyl group, such as 2-hydroxyethyl (meth)acrylate, 2-hydroxypropyl (meth)acrylate, 2-hydroxybutyl (meth)acrylate, 4-hydroxybutyl (meth)acrylate, 6-hydroxyhexyl (meth)acrylate, 8-hydroxyoctyl (meth)acrylate or the like.
(iv) (Meth)acrylic acid esters having a polyvalent hydroxy group, such as (meth)acrylic acid monoester of glycerin, (meth)acrylic acid monoester of trimethylolpropane or the like.
(v) Unsaturated carboxylic acids such as acrylic acid, methacrylic acid, maleic acid, itaconic acid or the like.
(vi) Unsaturated amides such as N-methylol acrylamide, diacetone acrylamide, dimethylaminopropyl acrylamide or the like.
(vii) (Meth)acrylic acid esters having an epoxy group, such as glycidyl (meth)acrylate or the like.
(viii) (Meth)acrylic acid esters having a carboxy group, such as 2-carboxyethyl (meth)acrylate or the like.
(ix) Vinyl acetate, (meth)acrylonitrile, and the like.

### [Structure of Copolymer]

In the copolymer, the molar ratio of units derived from the cationic functional group-containing monomer to units derived from the aromatic group-containing monomer may be 1:10 to 10:1, 1:8 to 8:1, or 1:5 to 5:1, preferably 1:4 to 4:1, more preferably 1:2 to 2:1, even more preferably 1.5:1 to 1:1.5, still even more preferably 1.2:1 to 1:1.2, particularly preferably 1.1:1 to 1:1.1, and most preferably 1:1.

### [Method of Producing Copolymer]

The copolymer can be obtained by mixing the cationic functional group-containing monomer and the aromatic group-containing monomer in a predetermined solvent in the presence of a polymerization initiator to carry out solution polymerization. As the cationic functional group-containing monomer and the aromatic group-containing monomer, those monomers in which R^{11a} in general formula (Ia) and R^{11b} in general formula (Ib) are the same are used.

Furthermore, by mixing a cationic functional group-containing monomer and an aromatic group-containing monomer in a predetermined solvent prior to the polymerization reaction, the cationic functional group-containing monomer units and the aromatic group-containing monomer units are likely to be arranged alternately due to the interaction between the cationic functional group and the aromatic group, thereby making it possible to increase the proportion of the above-described structure (I) in the copolymer.

When other monomers are used in addition to the cationic functional group-containing monomer and the aromatic group-containing monomer, they may be mixed before or after mixing the cationic functional group-containing monomer and aromatic group-containing monomer.

The temperature during the mixing may be, for example, 19° C. or higher and 35° C. or lower.

The molar ratio of the cationic functional group-containing monomer to be used to the aromatic group-containing monomer to be used may be 1:10 to 10:1, 1:8 to 8:1, or 1:5 to 5:1, preferably 1:4 to 4:1, more preferably 1:2 to 2:1, even more preferably 1.5:1 to 1:1.5, still even more preferably 1.2:1 to 1:1.2, particularly preferably 1.1:1 to 1:1.1, and most preferably 1:1.

The amount of the other monomers to be used may be determined within a range that does not prevent the cationic functional group-containing monomer units and the aromatic group-containing monomer units from being arranged alternately. Specifically, the amount of the other monomers to be used may be 50 mol % or less, or 30 mol % or less, preferably 10 mol % or less, more preferably 5 mol % or less, even more preferably 1 mol % or less, and particularly preferably 0 mol %, with respect to the total molar amount of the cationic functional group-containing monomer and the aromatic group-containing monomer.

The polymerization initiator may be any compound that initiates a polymerization reaction by heat or light, and examples thereof include 2-oxoglutaric acid, benzoyl peroxide, azobisisobutyronitrile, potassium persulfate, sodium persulfate and the like.

The amount of the polymerization initiator to be used may be 0.001 mol % or more and 0.100 mol % or less, 0.005 mol % or more and 0.050 mol % or less, or 0.010 mol % or more and 0.030 mol % or less, with respect to the total molar amount of the monomers used in the polymerization reaction.

The organic solvent used in the solution polymerization is preferably dimethyl sulfoxide (DMSO).

The amount of the organic solvent to be used is preferably an amount such that the total concentration of the monomers used in the polymerization reaction is 1 mol/L or more. The upper limit of the total concentration of the monomers in the reaction solution may be set to a level that does not cause the reaction solution to become too viscous, and may be set to, for example, 2 mol/L.

The polymerization reaction is initiated by heating the reaction solution to a temperature of, for example, about 50° C. to 100° C., or by irradiating the reaction solution with light such as UV light.

The polymerization reaction temperature can be appropriately set depending on the types of the monomers and the polymerization initiator to be used, and may be set to, for example, 20° C. or higher and 100° C. or lower.

The polymerization reaction time may be from 1 hour to 12 hours.

After the polymerization reaction, the copolymer may be purified.

The copolymer can be purified by a known method, and if necessary, a post-treatment may be performed to extract the copolymer. Specifically, if necessary, post-treatment operations such as filtration, washing, extraction, pH adjustment, dehydration, and concentration may be performed alone or in combination, and the copolymer can be purified by concentration, reprecipitation, column chromatography or the like.

The structure of the copolymer can be confirmed by known techniques such as nuclear magnetic resonance (NMR) spectroscopy, infrared spectroscopy (IR) or the like.

In order to remove as much of the organic solvent, which is toxic to living organisms, as possible from the obtained copolymer, it is preferable to replace the solvent with water by dialysis or the like.

After purification or solvent replacement of the copolymer, drying may be performed. Examples of the drying methods include ventilation drying, drying in a thermostatic chamber, drying under reduced pressure, hot air circulation drying, freeze drying and the like. Among these, freeze drying is preferred. When the freeze drying is performed, a cryoprotectant may be further included from the viewpoint of more effectively suppressing the increase in particle size of the microparticles formed by the copolymer.

The cryoprotectant is not particularly limited as long as it is known as a “cryoprotectant” or a “lyoprotectant for lyophilization”, and examples thereof include disaccharides, sorbitol, dextran, polyethylene glycol, propylene glycol, glycerin, glycerol, polyvinylpyrrolidone, dimethyl sulfoxide and the like.

The disaccharides are not particularly limited, and examples thereof include sucrose, lactulose, lactose, maltose, trehalose, cellobiose, kojibiose, nigerose, isomaltose, isotrehalose, neotrehalose, sophorose, laminaribiose, gentiobiose, turanose, maltulose, palatinose, gentiobiulose, mannobiose, melibiose, melibiulose, neolactose, galactosucrose, scillabiose, neohesperidose, rutinose, rutinulose, vicianose, xylobiose, primeverose and the like.

The amount of the cryoprotectant to be used is not particularly limited, and can be appropriately determined by those skilled in the art according to known methods.

The copolymer may be in solid, semi-solid or liquid form.

In the case of a solid, examples of the form thereof include powder, granules, tablets and the like. Among these, the solid is preferably a freeze-dried powder.

In the case of a semi-solid, examples of the form thereof include a gel.

In the case of a liquid, examples of the form thereof include an aqueous solution in which the powder is dissolved in water or a suspension in which the powder is suspended.

When the embolic agent of the present embodiment is a liquid, the concentration of the copolymer may be 10 mg/L or more and 100 mg/L or less, preferably 20 mg/L or more and 70 mg/L or less, more preferably 30 mg/L or more and 50 mg/L or less. When the concentration of the copolymer is equal to or more than the lower limit, it is possible to more efficiently form an embolism by interacting with blood. On the other hand, when the concentration is equal to or less than the upper limit, it is possible to more effectively store the embolic agent in a stable state by suppressing the increase in viscosity even when the embolic agent is stored for a long period of time, such as about one month. In addition, as shown in the examples described later, when administered to a living body, the embolic agent has excellent injectability by a syringe or a microcatheter, and can be safely extracted from a blood vessel with almost no resistance.

Examples of the subjects to which the embolic agent of the present embodiment is administered include, but are not limited to, humans, monkeys, dogs, cows, horses, sheep, pigs, rabbits, mice, rats, guinea pigs, hamsters and the like. Among these, mammals are preferred, and humans are particularly preferred.

Administration to the human or animal patient is preferably a local administration to the site where embolization is desired, as the copolymer interacts with the blood to form a gel.

Specific examples of the administration method include methods which are known to those skilled in the art and can be performed using a syringe, a microcatheter and the like, such as intraarterial injection, intravenous injection or the like.

The dosage varies depending on the patient's weight, age, symptoms, administration method and the like, and a person skilled in the art can appropriately select an appropriate dosage. The embolic agent of the present embodiment is administered in the form of an injection agent, with the copolymer concentration preferably being 20 mg/L or more and 60 mg/L or less.

The dosage of the commercially available Onyx is generally limited to about 4.5 mL or less per day in an adult (body weight 60 kg) due to the toxicity of the solvent DMSO. However, the embolic agent of the present embodiment is water-soluble and can be administered without using DMSO. Therefore, the embolic agent of the present embodiment can be administered in a larger dosage than Onyx, for example, more than about 4.5 mL per day in a typical adult (body weight 60 kg). Alternatively, when the required amount at the administration site is small, for example, the amount can be about 4.5 mL or less per day in a typical adult (body weight 60 kg).

The number of administrations may be a single administration of the above-mentioned dosage, or the above-mentioned dosage may be administered once every week, every two weeks, three weeks, four weeks, one month, two months, three months, or half a year, or may be administered twice or more times. Note that the embolic agent of the present embodiment is water-soluble and, unlike conventional embolic agents such as EVOH or the like, can be formulated to contain no organic solvents harmful to the body such as DMSO, so that the required dosage can be administered locally in a single dose.

### <<Pharmaceutical Composition>>

The embolic agent of the present embodiment can be used as a pharmaceutical composition in combination with a pharmaceutically acceptable carrier.

As the pharmaceutically acceptable carrier, those usually used in the preparation of pharmaceutical compositions can be used without any particular limitation. More specifically, for example, solvents for injections such as water, ethanol, glycerin and the like can be mentioned.

The pharmaceutical composition may further contain additives. Examples of the additives include lubricants such as calcium stearate, magnesium stearate or the like; stabilizers such as benzyl alcohol, phenol or the like; solubilizers such as benzyl benzoate, benzyl alcohol or the like; antioxidants; preservatives and the like.

The pharmaceutical composition can be formulated by appropriately combining the above-mentioned embolic agent with the above-mentioned pharmaceutically acceptable carriers and additives, and mixing them in a unit dosage form required for the generally accepted pharmaceutical practice.

The dosage form of the pharmaceutical composition is preferably an injection agent.

The pharmaceutical composition is preferably used for the treatment of diseases to which known embolic agents are applicable. Such diseases include cerebral arteriovenous malformation; uterine fibroids, brain tumors, and liver cancer (the embolic agent of the present embodiment can be applied to the arterial embolization for these diseases); cerebral aneurysm; dural arteriovenous fistula; coronary arteriovenous fistula; bleeding due to vascular damage caused by trauma; nosebleeds; obstetric bleeding (atonic bleeding, placental abruption) and the like.

### OTHER EMBODIMENTS

In one embodiment, the present invention provides a method for treating a disease, including administering to a patient in need of treatment a therapeutically effective amount of the above-described embolic agent.

The diseases include the same as those exemplified above in the pharmaceutical composition.

In one embodiment, the present invention also provides a use of the above-mentioned embolic agent for the manufacture of a pharmaceutical composition for the treatment of a disease.

In one embodiment, the above-mentioned embolic agent and contrast agent can be included in a kit for vascular embolization.

Examples of the contrast agent include gadolinium, Gd-DTPA, Gd-DTPA-BMA, Gd-HP-DO3A, iodine, iron, iron oxide, chromium, manganese, tantalum, and complexes and chelate complexes thereof.

### EXAMPLE

The present invention will be described below with reference to the Examples, but the present invention is not limited to the following Examples.

### Example 1

(Preparation of cationic a polymer (poly(cation-adj-π)))

The cationic π polymer (poly(cation-adj-π)) was prepared by dissolving a cationic functional group-containing monomer (0.5 M), an aromatic group-containing monomer (0.5 M), and 2-oxoglutaric acid (0.25 mM) as a photopolymerization initiator in DMSO, and irradiating the resulting mixture with 365 nm UV light (4 mW/cm²) at room temperature (approximately 25° C.) for 11 hours to copolymerize the monomers to obtain a cationic π polymer.

The combinations of the cationic functional group-containing monomer and the aromatic group-containing monomer used are as shown in the table below.

TABLE 1

| Cationic functional group-containing | Aromatic group-containing |
|---|---|
| monomer | monomer |
| 2-(acryloyloxy)ethyl | Benzyl acrylate (BZA) |
| trimethylammonium | 2-phenoxyethyl acrylate (PEA) |
| chloride (ATAC) | 2-(2-phenoxyethoxy)ethyl acrylate |
| | (PDEA) |
| | 2-(phenylsulfanyl)ethyl |
| | acrylate (PSEA) |
| 2-(methancryloyloxy)ethyl | 2-phenoxyethyl methacrylate |
| trimethylammonium chloride | 2-hydroxy-3-phenoxypropyl |
| | methancrylate |

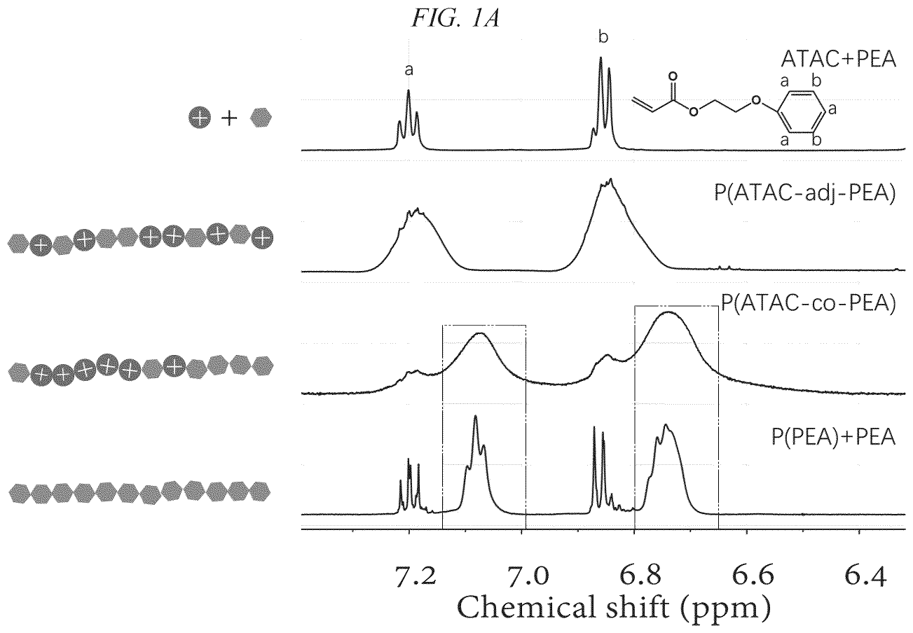

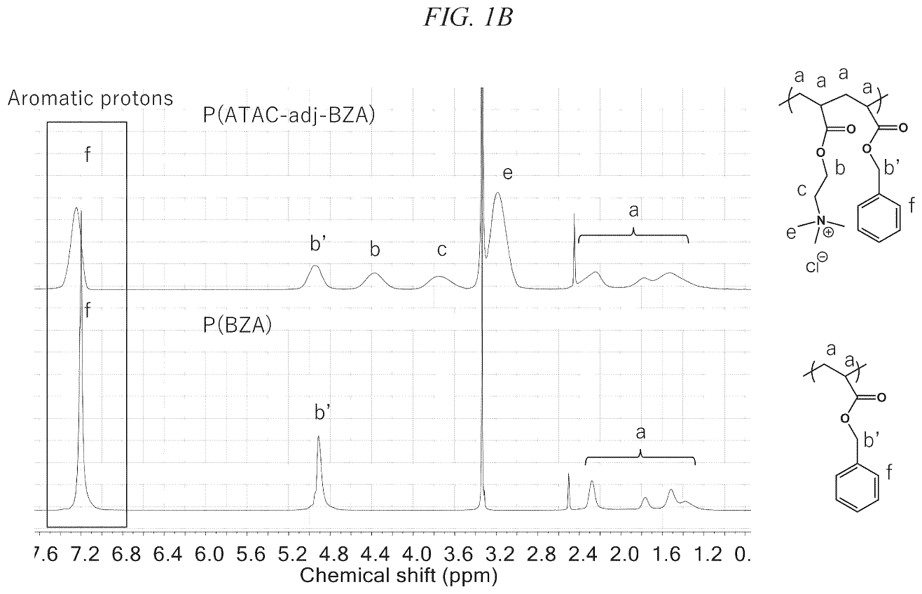

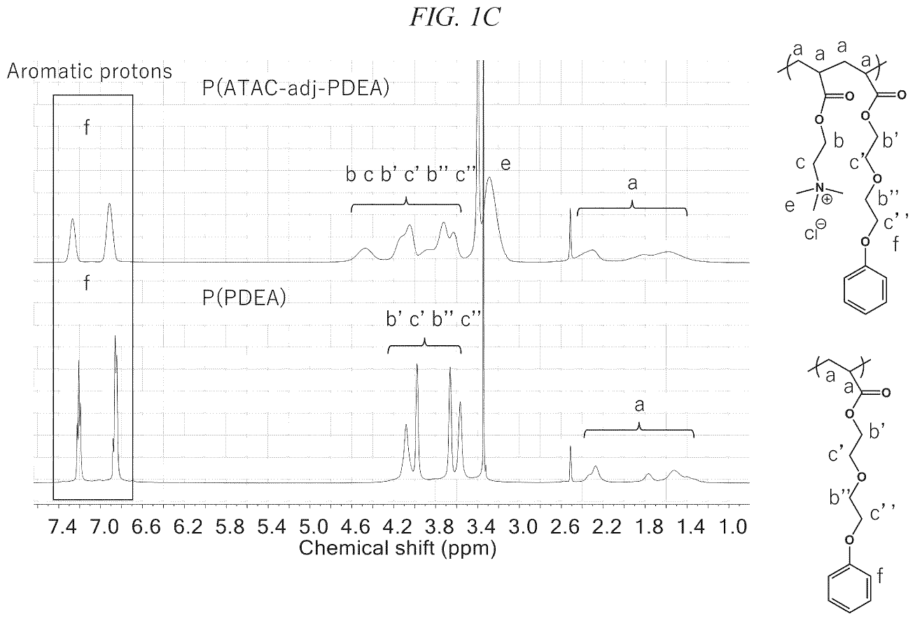

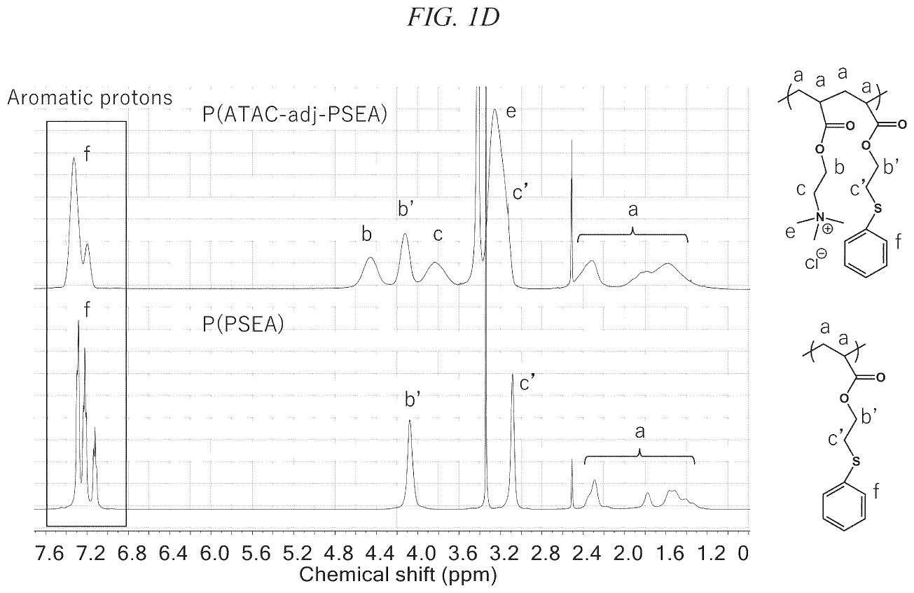

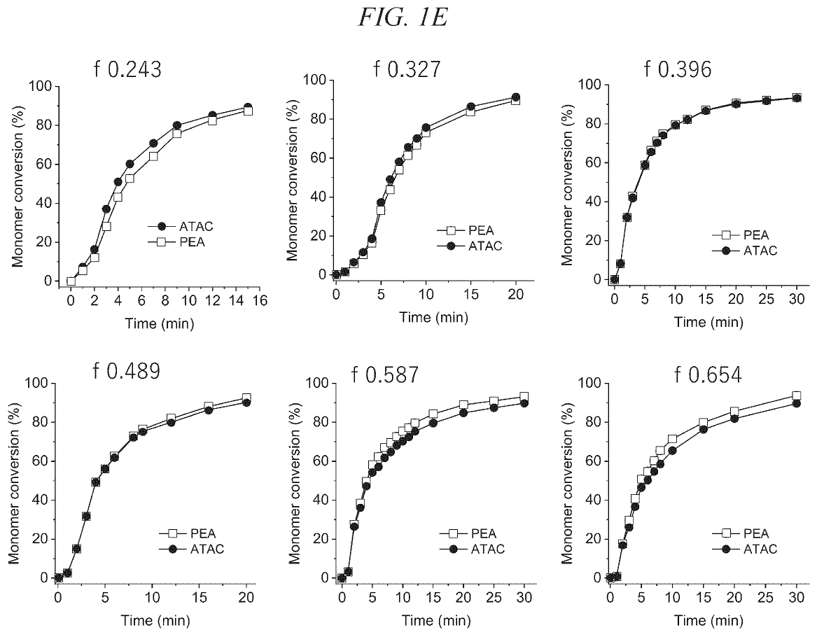

As a representative result, the NMR spectrum of the cationic π-polymer (P(ATAC-adj-PEA)) in a state dissolved in DMSO prepared using ATAC and PEA is shown in FIG. 1A. In FIG. 1A, P(ATAC-co-PEA) is a random copolymer of ATAC and PEA, which was prepared by dissolving a cationic functional group-containing monomer (0.5 M), an aromatic group-containing monomer (0.5 M), and 2-oxoglutaric acid (0.25 mM) as a photopolymerization initiator in dimethyl sulfide (DMS), and irradiating the resulting mixture with 365 nm UV light at room temperature (about 25° C.) for 11 hours to cause random copolymerization. P(PEA) is a homopolymer obtained by homopolymerizing PEA. The circled + indicates ATAC, and the hexagon indicates PEA.

As for the measurement conditions for ¹H-NMR measurement, each polymer was dissolved in a deuterated DMSO (DMSO-_{d6}) solution at a concentration of 1 to 5 mg/mL, and analyzed using a ¹H-NMR (Agilent 500 MHz).

As shown in FIG. 1A, in P(ATAC-adj-PEA), the phenyl proton signals showed symmetric broadening around the phenyl proton peaks of the aromatic group-containing monomer, indicating that the cationic functional group and the aromatic group were distributed adjacently on the polymer chain, whereas in P(ATAC-co-PEA), the phenyl proton signals had new broad peaks at high magnetic field, similar to the signals of the homopolymer of the aromatic group-containing monomer, indicating that the aromatic group-containing monomer was homopolymerized into long segments of the polymer chain.

In addition, the NMR spectra of each P(ATAC-adj-BZA), P(ATAC-adj-PDEA), and P(ATAC-adj-PSEA) is shown in FIG. 1B to FIG. 1D.

As shown in FIGS. 1B to 1D, in P(ATAC-adj-BZA), P(ATAC-adj-PDEA), and P(ATAC-adj-PSEA), the phenyl proton signals were located at higher chemical shifts than those of the homopolymer of the aromatic group-containing monomer. Furthermore, in P(ATAC-adj-BZA), P(ATAC-adj-PDEA), and P(ATAC-adj-PSEA), the phenyl proton signal peaks showed broader shapes than those of the homopolymer of the aromatic group-containing monomer. These differences indicate that the cationic functional groups and aromatic groups were adjacently distributed on the polymer chain in P(ATAC-adj-BZA), P(ATAC-adj-PDEA), and P(ATAC-adj-PSEA).

Furthermore, P(ATAC-adj-PEA) was prepared in the same manner as described above, with a total monomer concentration of 1.0 M, using 0.25 mM of 2-oxoglutaric acid as a polymerization initiator, and DMSO as a solvent, and adjusting the molar ratio of ATAC to PEA. The copolymerization rate of the cationic functional group-containing monomer (ATAC) and the aromatic group-containing monomer (PEA) was determined by analyzing the polymers at different reaction times using a ¹H-NMR (Agilent 500 MHz). The results are shown in FIG. 1E. In FIG. 1E, “f” indicates the molar ratio of PEA. For example, in “f 0.243”, 0.757M of ATAC and 0.243M of PEA were used.

As shown in FIG. 1E, polymers were formed at similar rates in the range of ATAC:PEA=0.757M:0.243M to 0.346M:0.654M, confirming that P(ATAC-adj-PEA) can be produced within the above-described molar ratio range.

Next, the solvent of each of the obtained cationic 2-polymers was changed from DMSO to distilled water by dialysis, and the polymer was freeze-dried (kept at −40° C. under vacuum (<30 Pa) for several hours to several days depending on the amount), and then dissolved in water to prepare polymer aqueous solutions at 30 mg/mL, 40 mg/mL, and 50 mg/mL.

### Example 2

### (Qualitative and Quantitative Blood Agglomeration Tests)

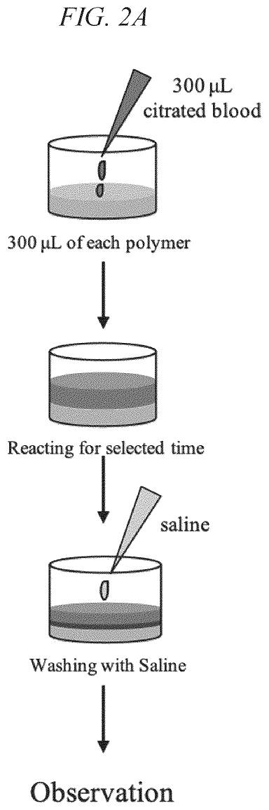

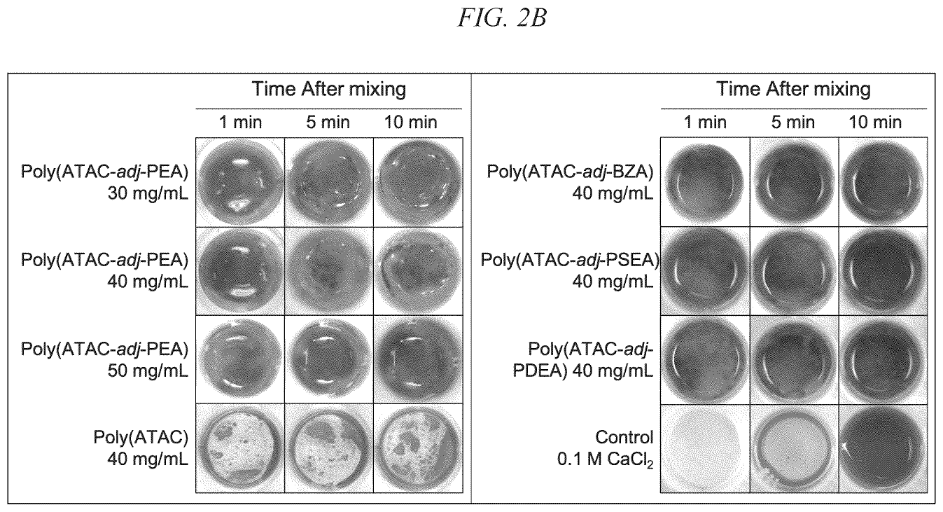

For the qualitative blood agglomeration test, 150 μL of polymer solutions with different concentrations (30 mg/mL, 40 mg/mL, or 50 mg/mL) were each added to a well plate to cover the entire bottom surface with the polymer. Then, the same volume of citrated blood was added to each well. The wells were repeatedly washed with saline until the solution was clear to remove all non-agglutinated blood components (see FIG. 2A). 150 μL of citrated blood containing 0.1M calcium chloride (CaCl₂) was used as a control. The results are shown in FIG. 2B.

As shown in FIG. 2B, the blood gels formed using each cationic π-polymer were stable even when washed with saline, whereas the blood gels formed using the ATAC homopolymer collapsed and flowed out when washed with saline.

The above-described results demonstrates that the cationic π-polymer can form a stable gel with blood under physiological conditions.

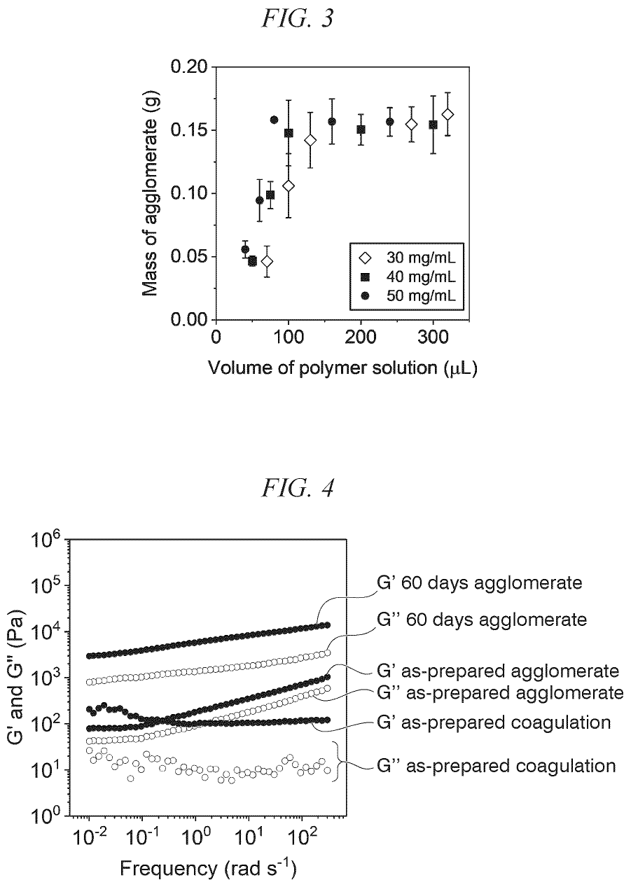

Subsequently, a quantitative blood agglomeration test was carried out as follows. First, 500 μL of citrated blood was first added to a microcentrifuge tube, and different amounts (42 μL to 320 μL) of polymer (P(ATAC-adj-PEA)) aqueous solutions with different concentrations (30 mg/mL, 40 mg/mL, or 50 mg/mL) were slowly added to the blood. After injection, the agglomerates were immediately taken out and the liquid on the surface was removed. Then, the weight of the agglomerates was measured. The results are shown in FIG. 3.

As shown in FIG. 3, the mass of the blood gel was almost constant at any concentration when the amount of the polymer aqueous solution was 100 μL or more. In other words, it was revealed that a saturated mass of blood gel exists at a certain amount of blood.

Rheological tests were performed on the agglomerates obtained by adding 600 L of a 40 mg/mL aqueous solution of the polymer (P(ATAC-adj-PEA)) to 1 mL of citrated blood using an ARES-G2 rheometer (TA Instruments). A control coagulation was obtained by adding 150 μL of a 0.1M aqueous solution of calcium chloride (CaCl₂) to 500 L of citrated blood. The results are shown in FIG. 4. In FIG. 4, “as-prepared coagulation” indicates the control coagulation, “as-prepared agglomerate” indicates the blood gel formed using the aqueous solution of the polymer (P(ATAC-adj-PEA)), and “60 days agglomerate” indicates the blood gel stored in physiological saline at 37° C. (exchanged daily) for 60 days. The vertical axis of the graph indicates shear stress (Pa), “G′” indicates storage modulus (spring elasticity), and “G″” indicates loss modulus (viscous part). The horizontal axis indicates frequency (rad/s).

As shown in FIG. 4, it was revealed that the blood gel is soft, has viscoelasticity, and is stable for a long period of time.

### Example 3

### (Injection Test)

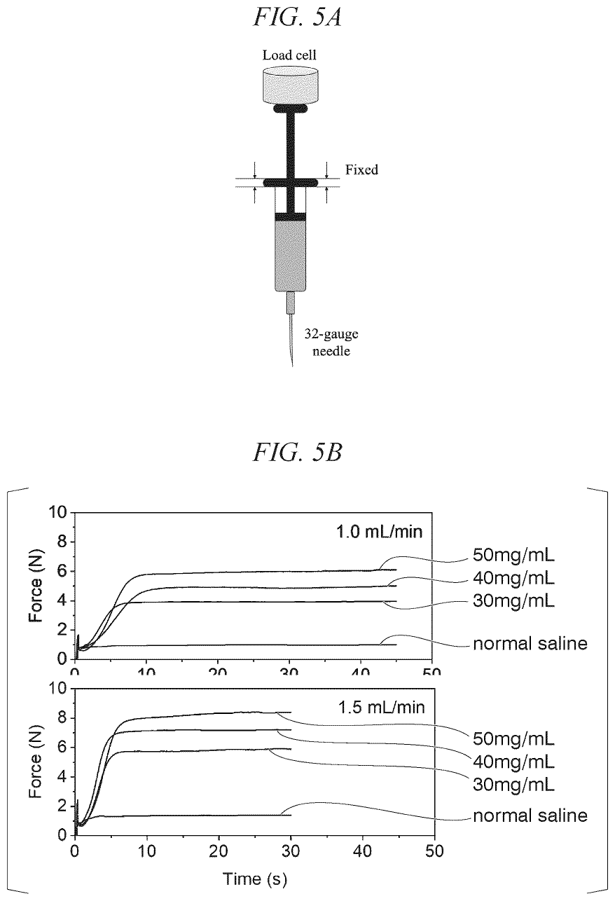

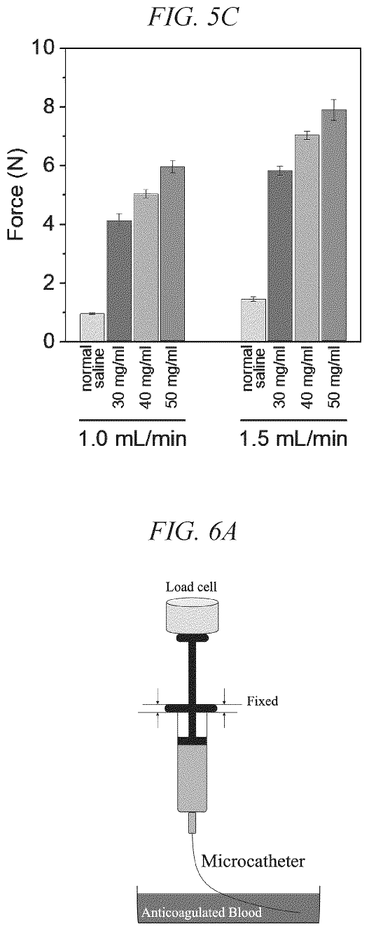

An injection force test was conducted using a 1 mL plastic syringe (see FIG. 5A). The syringe (needle size: 32 gauge (G), I.D. 0.26 mm) was fixed to a tester so that the syringe would not move during the test, and injection was performed at a rate of 1 mL/min and 1.5 mL/min, respectively, to measure the injection pressure (N). In the needle injection test, 1 mL of an aqueous solution of the polymer (P(ATAC-adj-PEA)) with different concentrations (30 mg/mL, 40 mg/mL, or 50 mg/mL) was added to the syringe and injected. A similar test was conducted using saline as a control. The results are shown in FIG. 5B and FIG. 5C.

As shown in FIG. 5B and FIG. 5C, it was confirmed that the polymer (P(ATAC-adj-PEA)) aqueous solution of any concentration could be smoothly injected with a relatively small force of about 4N or more and 8N or less.

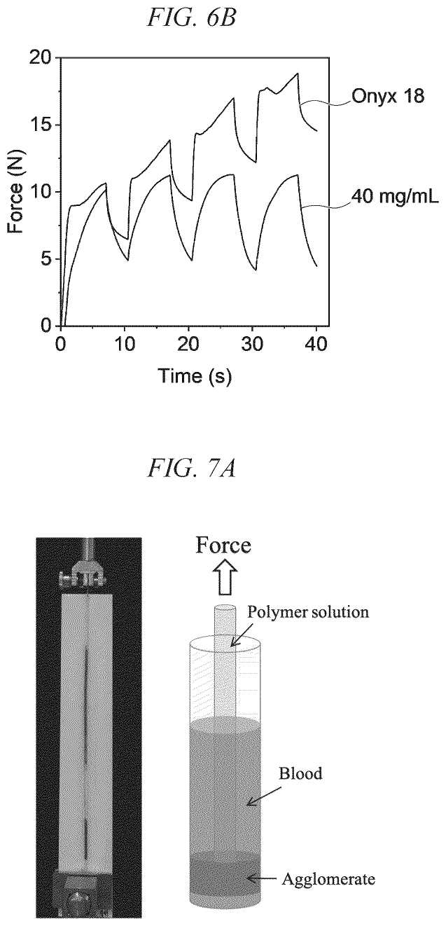

Next, an injection test was also conducted using a 1 mL plastic syringe and a microcatheter (see FIG. 6A). A polymer (P(ATAC-adj-PEA)) aqueous solution (40 mg/mL) containing tantalum powder (0.25 mg/mL) was injected from the syringe through a 150 cm long clinically used microcatheter (I.D. 0.017 inch (0.43 mm)) toward anticoagulated blood at a rate of 1 mL/min, with 7 seconds of injection and 3 seconds of pause repeated 4 times, and the injection pressure (N) was measured. A similar test was conducted using a conventional embolic material, EVOH (trade name “Onyx (registered trademark)” manufactured by ev3 Endovascular) as a control. The results are shown in FIG. 6B.

As shown in FIG. 6B, with Onyx, the injection pressure tended to increase with the number of injection/pause cycles. On the other hand, with the polymer (P(ATAC-adj-PEA)) aqueous solution, it was confirmed that the polymer (P(ATAC-adj-PEA)) aqueous solution can be smoothly injected at an injection pressure of 1 IN or less using a microcatheter without increasing the injection pressure, even when the injection/pause cycle was repeated.

### Example 4

### (Traction Test)

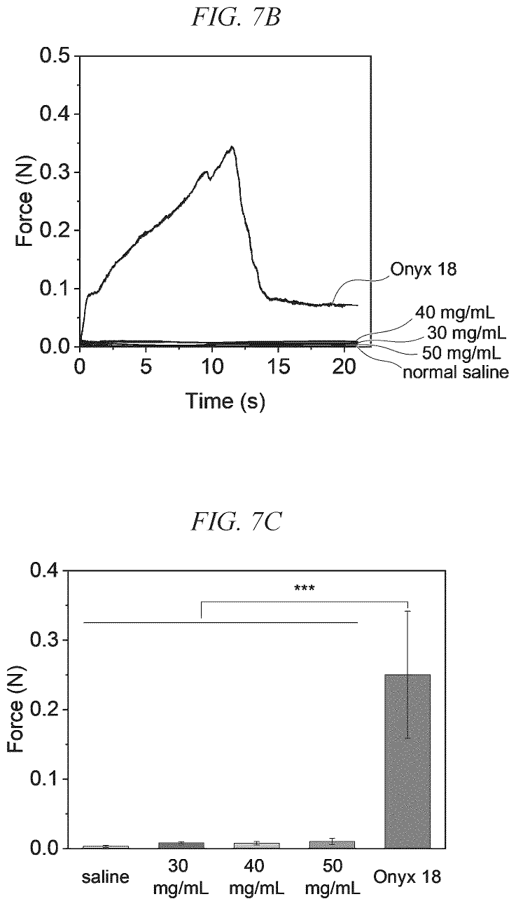

A polyethylene tube (ID 0.28 mm, OD 0.61 mm) filled with a polymer (P(ATAC-adj-PEA)) aqueous solution of different concentrations (30 mg/mL, 40 mg/mL, or 50 mg/mL) was passed through a thick polyethylene tube (ID 1.4 mm, OD 1.9 mm) containing 0.1 mL of citrated blood, and the same amount of each polymer solution was injected. After leaving it for 30 minutes, the force (N) when the thin polyethylene tube was withdrawn at a speed of 1.0 mm/min was measured (see FIG. 7A). A similar test was performed using a conventional embolic material EVOH (trade name “Onyx (registered trademark)” manufactured by ev3 Endovascular) and physiological saline as controls. The results are shown in FIG. 7B and FIG. 7C.

As shown in FIG. 7B and FIG. 7C, in the case of Onyx, due to the fixation by adhesion, a force of about 0.25N was required to withdrawn it. On the other hand, in the case of the polymer (P(ATAC-adj-PEA)) aqueous solution, there was no fixation by adhesion after reaction with blood, and the withdrawal force was approximately the same as that of physiological saline.

### Example 5

### (In Vivo Safety Test)

Under anesthesia, a 1 cm long skin incision was made on the right dorsal side of an 8-week-old male Sprague-Dawley rat to create a subcutaneous pocket, and a 40 mg/mL polymer (P(ATAC-adj-PEA)) hydrogel disk (diameter 5 mm, thickness 1 mm) was placed in the subcutaneous pocket.

The hydrogel of the polymer (P(ATAC-adj-PEA)) was prepared by the following method and then cut to the above-described size. First, monomers (ATAC 1.2M and PEA 1.2M) and 2-oxoglutaric acid (6 mM) as a polymerization initiator were dissolved in DMSO. Next, the mixture was poured into a reaction cell consisting of a pair of glass plates. Polymerization was carried out by irradiating the plate with 365 nm UV light (4 mW/cm²) at 1 mm intervals at room temperature (about 25° C.) for 11 hours in a glove box. After polymerization, the prepared hydrogel was immersed in a large amount of saline to wash away DMSO and residual monomers, and a hydrogel was obtained.

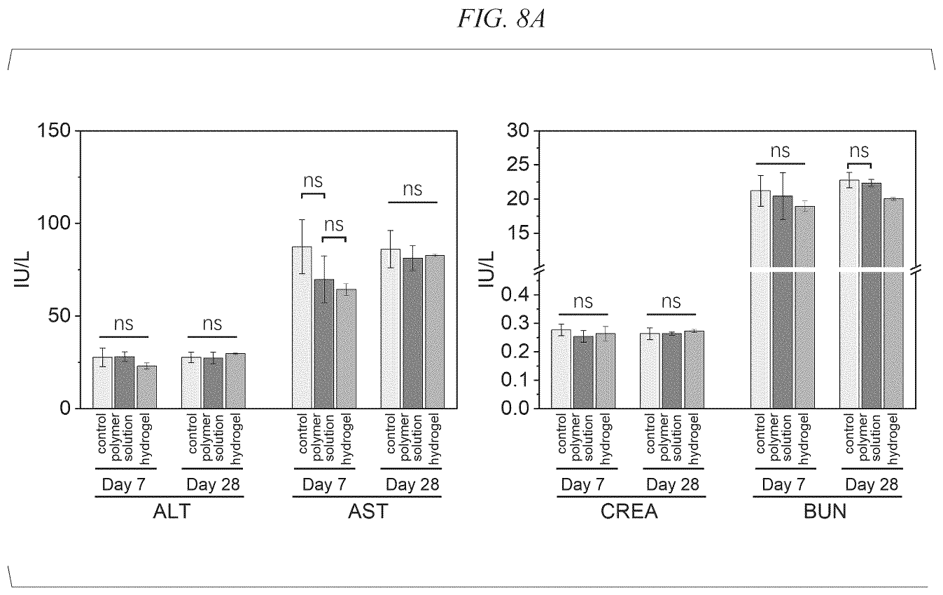

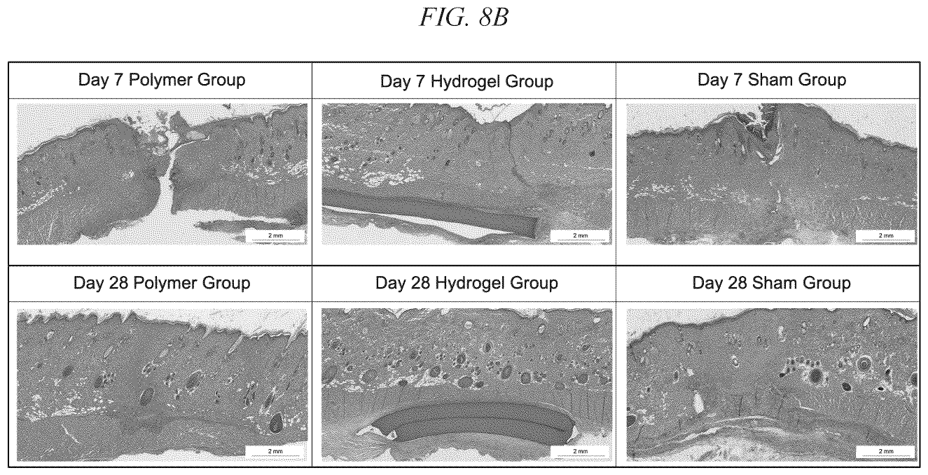

The incision was then sutured. Rats that underwent only surgical incision served as controls. Blood biochemistry tests were performed on the 7th and 28th days after surgery. The rats' venous blood was centrifuged to obtain serum for testing. The concentrations of alanine aminotransferase (ALT), aspartate aminotransferase (AST), creatinine (CREA), and urea nitrogen (BUN) in serum were measured by a specialized laboratory (SRL, Inc., Tokyo, Japan). In general, the amounts of ALT and AST in blood are indicators of liver function, and the amounts of CREA and BUN in blood are indicators of kidney function. The results are shown in FIG. 8A.

As shown in FIG. 8A, even on the 28th day after surgery, the levels of ALT and AST in the blood, as well as the levels of CREA and BUN in the blood, were similar to those in the control, and no abnormalities were observed in liver function or kidney function.

In addition, after the rats were euthanized on the 7th and 28th days after surgery, tissue sections were prepared from the area where 40 mg/mL polymer (P(ATAC-adj-PEA)) was injected, the area where the hydrogel was implanted, and the area where the surgical incision was made, and histological analysis (hematoxylin and eosin (H&E) staining) was also performed. The results are shown in FIG. 8B. In FIG. 8B, the scale bar is 2 mm.

As shown in FIG. 8B, no severe inflammatory reaction was observed at the transplant site after transplantation.

### Example 6

### (Intravascular Administration Test)

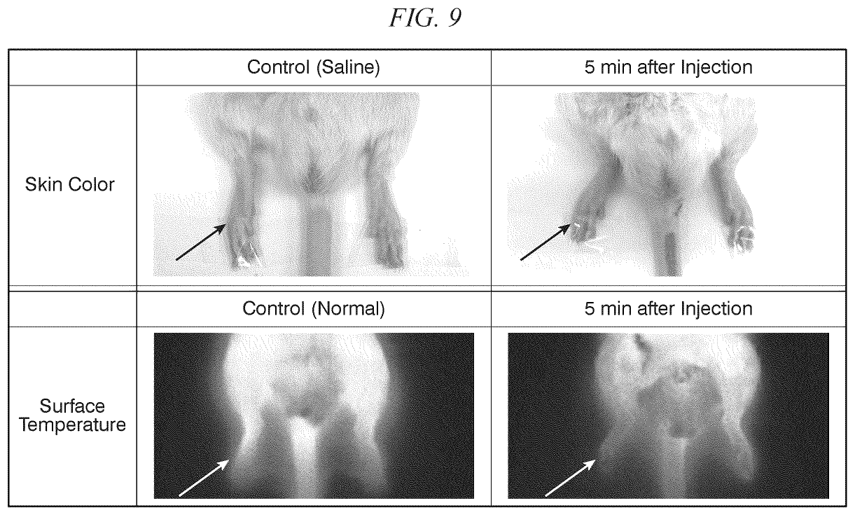

Under anesthesia, a 1 cm longitudinal incision was made in the fore thigh of the right hind leg of 8-week-old male Sprague-Dawley rats, the femoral artery was exposed under a surgical microscope, and then 0.1 mL of 40 mg/mL polymer (P(ATAC-adj-PEA)) aqueous solution was injected distal to the artery using a 32-gauge needle. The incision was then closed, and the rats were allowed to recover from the anesthesia. Rats injected with the same amount of saline served as controls. Five minutes after surgery, the skin color of the hind legs was visually observed. In addition, the surface temperature of the hind legs was measured using a thermograph (Optris, PI640i). The results are shown in FIG. 9.

As shown in FIG. 9, 5 minutes after the operation, the skin color of the hind limbs changed and the surface temperature also decreased, confirming the embolism.

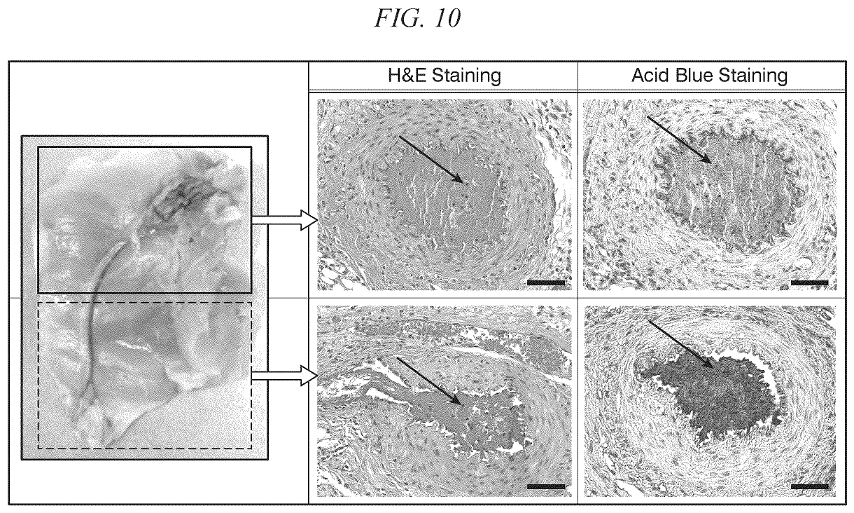

Five minutes after the operation, the rats were euthanized, and the right hind limbs were taken to prepare tissue sections and to perform histological analysis (H&E staining for tissue observation and acid blue staining for polymer observation). The results are shown in FIG. 10. In FIG. 10, the scale bar is 100 m. The arrows in the stained images indicate the nuclei of white blood cells stained with hematoxylin.

As shown in FIG. 10, it was confirmed that embolic material was formed from the blood components and the polymers.

### Example 7

### (Computed Tomography (CT) Imaging)

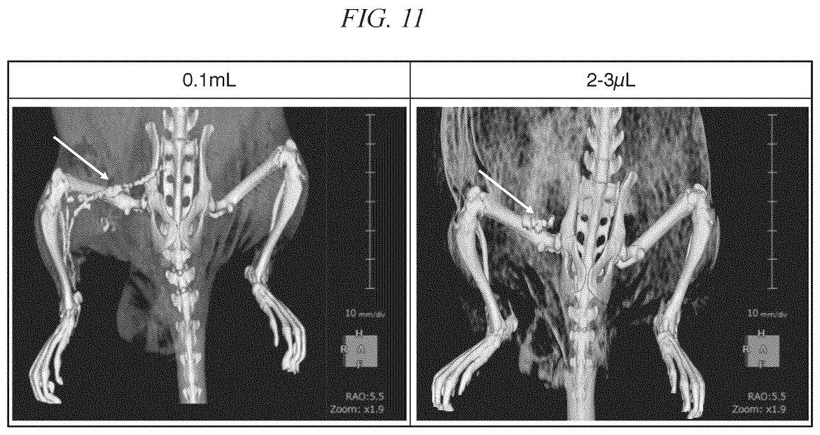

A 40 mg/mL aqueous solution of polymer (P(ATAC-adj-PEA)) containing tantalum powder (0.25 g/mL) was prepared prior to surgery and tested at different injection volumes (0.1 mL, or 2 μL or more and 3 μL or less). In the first test, 0.1 mL of the polymer aqueous solution containing tantalum powder was injected into the right femoral artery of 8-week-old male Sprague-Dawley rats within 5 seconds. 45 minutes after injection, a CT scan of the right hind limb was taken to confirm embolization. The results are shown in the left side of FIG. 11.

In the next test, the polymer aqueous solution containing tantalum powder was injected as a one-shot injection (2 μL or more and 3 μL or less) into the right femoral artery of 8-week-old male Sprague-Dawley rats using the same procedure as described above. Three hours after injection, a CT scan of the right hind limb was taken to confirm embolization. The results are shown on the right side of FIG. 11.

The CT image on the left side of FIG. 11 confirmed that the embolism had reached a distal location from the injection site.

The CT image on the right side of FIG. 11 confirmed that the embolism has formed at the injection site.

These results demonstrate that by appropriately adjusting the injection amount and selecting the injection site, embolism can be formed at a desired site and in a desired area within the body.

### INDUSTRIAL APPLICABILITY

According to the embolic agent of the present embodiment, it is possible to provide an embolic agent that has vascular embolization properties equivalent to those of conventional embolic agents, while being easy to inject with a syringe and having excellent safety and stability in the living body.

1. An embolic agent comprising, as an active ingredient, a copolymer of a cationic functional group-containing monomer and an aromatic group-containing monomer, the copolymer having at least a part thereof a structure represented by the following general formula (I): 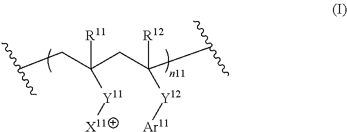
in general formula (I), R¹¹ and R¹² are hydrogen atoms or alkyl groups having 1 to 20 carbon atoms which may have a substituent, and satisfy a relational formula: R¹¹=R¹²; Y¹¹ and Y¹² are each independently a single bond, or an alkylene group having 1 to carbon atoms which may contain one or more selected from the group consisting of a hydroxyl group, an ester bond, an ether bond, a sulfide group, a carbonyl group, an amide bond and a phosphodiester bond; Ar¹¹ is an aromatic hydrocarbon group having 6 to 16 carbon atoms which may have a substituent; X¹¹ is an ammonium group; and n11 is an integer of 10 to 1,000.

2. The embolic agent according to claim 1, wherein in general formula (I), Ar11 is a phenyl group which may have a substituent.

3. The embolic agent according to claim 1, wherein in general formula (I), X11 is a quaternary ammonium group.

4. The embolic agent according to claim 1, wherein in general formula (I), an absolute value of a difference between a number of carbon atoms of Y12 and a number of carbon atoms of Y11 is 0 or more and 3 or less.

5. The embolic agent according to claim 1, wherein the structure represented by general formula (I) is a structure represented by the following general formula (I-1): 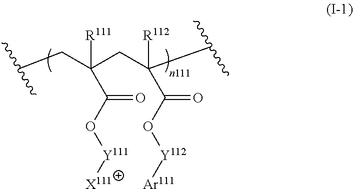
in general formula (I-1), R¹¹¹ and R¹¹² are hydrogen atoms or methyl groups, and satisfy a relational formula: R¹¹¹=R¹¹²; Y¹¹¹ and Y¹¹² are each independently an alkylene group having 1 to 20 carbon atoms which may contain one or more selected from the group consisting of a hydroxyl group, an ether bond, a sulfide group and a phosphodiester bond; Ar¹¹¹ is a phenyl group which may have a substituent; X¹¹¹ is a quaternary ammonium group; and n111 is an integer of 10 to 1,000.

6. The embolic agent according to claim 1, wherein in the copolymer, a molar ratio of a unit derived from the cationic functional group-containing monomer to a unit derived from the aromatic group-containing monomer is 1:4 to 4:1.

7. A kit for vascular embolization comprising the embolic agent according to claim 1 and a contrast agent.
